(19) United States
(12) Patent Application Publication
(10) Pub. No.: US 2019/0119649 A1
(43) Pub. Date: Apr. 25, 2019

(54) METHODS FOR GENERATING AUGMENTED STEM CELL-DERIVED BETA CELLS AND USES THEREOF

(71) Applicant: President and Fellows of Harvard College, Cambridge, MA (US)

(72) Inventors: Douglas A. Melton, Lexington, MA (US); Jeffrey R. Millman, St. Louis, MO (US); Mads Gürtler, København ø (DK)

(21) Appl. No.: 15/982,925

(22) Filed: May 17, 2018

Related U.S. Application Data
(63) Continuation of application No. 15/694,696, filed on Sep. 1, 2017, now abandoned.
Application No. 15/694,696 is a continuation of application No. 14/975,421, filed on Dec. 18, 2015, now abandoned.
(60) Provisional application No. 62/093,942, filed on Dec. 18, 2014.

(51) Int. Cl. C12N 5/071; G01N 33/50; A61K 35/39

(52) U.S. Cl. CPC C12N 5/0678; G01N 33/507; A61K 47/32; G01N 33/5073; A61K 35/39

(57) ABSTRACT

Disclosed herein are methods for generating augmented SC-β cells, and isolated populations of augmented SC-β cells for use in various applications, such as cell therapy.

### RELATED APPLICATIONS

This application is a continuation application of U.S. application Ser. No. 15/694,696, filed Sep. 1, 2017, which is a continuation application of U.S. application Ser. No. 14/975,421, filed Dec. 18, 2015, which claims the benefit of U.S. Provisional Application Ser. No. 62/093,942 filed on Dec. 18, 2014. The entire teachings of the above applications are incorporated herein by reference.

### BACKGROUND OF THE INVENTION

Diabetes affects more than 300 million people worldwide according to the International Diabetes Federation. Type 1 diabetes and type 2 diabetes involve β cell destruction and/or β cell dysfunction. Diabetic patients, particularly those suffering from type 1 diabetes, could potentially be cured through transplantation of β cells. While cadaveric human islet transplantation can render patients insulin independent for 5 years or longer, such approach is limited due to the scarcity and quality of donor islets (Bellin et al., 2012). Generating an unlimited supply of human β cells from stem cells could provide therapy to millions of patients as only a single cell type, the β cell, likely needs to be produced, and the mode of delivery is well understood: transplantation to a vascularized location within the body with immunoprotection. In addition, screening to identify novel drugs that improve β cell function, survival, or proliferation is also delayed due to limited islet supply and variability resulting from different causes of death, donor genetics, and other aspects in their isolation. As such, a steady, uniform supply of stem-cell-derived β cells would offer a useful drug discovery platform for diabetes. Moreover, genetically diverse stem-cell-derived β cells could be used for disease modeling in vitro or in vivo.

### SUMMARY OF THE INVENTION

There is a need for methods of generating augmented stem cell-derived β (SC-β) cells. The present invention is directed toward solutions to address this need, in addition to having other desirable characteristics.

In accordance with an embodiment of the present invention, a method for generating augmented stem cell-derived β(SC-β) cells is provided. The method includes contacting a cell population comprising SC-β cells, or precursors thereof, with an effective amount of an agent that decreases the level and/or activity of AXL receptor tyrosine kinase (AXL) and an antioxidant for a period of time sufficient for the level of MAFA gene expression to increase in the SC-β cells to at least 2 fold greater than the level of MAFA gene expression in the SC-β cells in the absence of contact with the agent and the antioxidant, thereby generating augmented SC-β cells.

In accordance with aspects of the present invention, the level of MAFA gene expression in the augmented SC-β cells is at least 10 times greater than the level of MAFA gene expression in the SC-β cells.

In accordance with aspects of the present invention, the cell precursors are selected from the group consisting of pluripotent stem cells, SOX17+ definitive endoderm cells, PDX1+ primitive gut tube cells, PDX1+/NKX6.1+ pancreatic progenitor cells, PDX1+/NKX6.1+/NEUROD1+ endocrine progenitor cells, PDX1+/NKX6.1+/NEUROD+/insulin+/glucagon−/somatostatin− cells, and combinations thereof.

In accordance with aspects of the present invention, the agent comprises R428. In accordance with aspects of the present invention, the effective amount of the agent comprises a concentration of 2 μM.

In accordance with aspects of the present invention, the antioxidant is selected from the group consisting of N-acetylcysteine, ascorbic acid, vitamin E, disodium 4,5-dihydroxy-1,3-benzenedisulfonate (Tiron). In accordance with aspects of the present invention, the effective amount of the antioxidant comprises a concentration of 1 mM.

In accordance with aspects of the present invention, the period of time comprises between 7 days and 21 days.

In accordance with aspects of the present invention, between at least 5% and 65% of the SC-β cells in the population become augmented SC-β cells.

In accordance with an embodiment of the present invention, an isolated augmented SC-β cell or population thereof generated according to the methods described herein is provided. The isolated augmented SC-β cell or population thereof exhibits a glucose stimulated insulin secretion (GSIS) response both in vitro and in vivo.

In accordance with aspects of the present invention, an isolated augmented SC-β cell or population thereof exhibits a stimulation index that is at least between 2.3-fold and 2.9-fold greater than the stimulation index of a control SC-β cell.

In accordance with aspects of the present invention, an isolated augmented SC-β cell or population thereof produces between approximately 300 uIU and 4000 uIU per 30 minute incubation at a high glucose concentration.

In accordance with an embodiment of the present invention, a microcapsule comprising the isolated augmented SC-β cell or population encapsulated therein is provided.

In accordance with an embodiment of the present invention, a cell line comprising the isolated augmented SC-β cell that stably expresses insulin is provided.

In accordance with an embodiment of the present invention, assays comprising the isolated augmented SC-β cell, or population thereof, or the cell line are provided.

The assays can be used for: i) identifying one or more candidate agents which promote or inhibit a β cell fate selected from the group consisting of β cell proliferation, β cell replication, β cell death, β cell function, β cell susceptibility to immune attack, and β cell susceptibility to dedifferentiation or differentiation; or ii) identifying one or more candidate agents which promote the differentiation of at least one insulin-positive endocrine cell or a precursor thereof into at least one SC-β cell.

In accordance with an embodiment of the present invention, a method for the treatment of a subject in need thereof is disclosed. The method includes administering to a subject in need thereof i) an isolated population of augmented SC-β cells, ii) a microcapsule comprising SC-β cells encapsulated therein; and/or iii) a macroencapsulation device comprising the SC-β cells encapsulated therein. In accordance with an embodiment of the present invention, an isolated population of augmented SC-β cells, a microcapsule comprising the isolated population of augmented SC-β cells, and/or a macroencapsulation device comprising the isolated population of augmented SC-β cells is used for administering to a subject in need thereof. In accordance with aspects of the invention, the subject has, or has an increased risk of developing diabetes or has, or has an increased risk of developing a metabolic disorder.

In accordance with an embodiment of the present invention, an artificial islet or pancreas comprising augmented SC-β cells is provided.

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3rd ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange; 10th ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, World Wide Web URL: ncbi.nlm.nih.gov/omim/and in Online Mendelian Inheritance in Animals (OMIA), a database of genes, inherited disorders and traits in animal species (other than human and mouse), at omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

### BRIEF DESCRIPTION OF THE DRAWINGS

These and other characteristics of the present invention will be more fully understood by reference to the following detailed description in conjunction with the attached drawings. The patent or application file contains at least one drawing executed in color. Copies of this patent or patent application publication with color drawings will be provided by the Office upon request and payment of the necessary fee.

FIG. 1A is a graph demonstrating that SC-β cells generated by contacting SC-β cells (or precursors thereof (e.g., endocrine progenitor cells) directed to differentiate into SC-β cells) with an exemplary agent that decreases the level and/or activity of AXL receptor tyrosine kinase (AXL), e.g., AXL inhibitor R428, and an antioxidant, e.g., N-acetylcysteine, exhibit increased MAFA gene expression relative to SC-β cells generated using the same protocol in the absence of treatment with the AXL inhibitor and the antioxidant.

FIG. 1B and FIG. 1C are graphs demonstrating that SC-β cells generated by contacting SC-β cells (or precursors thereof (e.g., endocrine progenitor cells) directed to differentiate into SC-β cells) with an exemplary agent that decreases the level and/or activity of AXL receptor tyrosine kinase (AXL), e.g., AXL inhibitor R428, and an antioxidant, e.g., N-acetylcysteine, exhibit a greater stimulation index relative to SC-β cells generated using the same protocol in the absence of treatment with the AXL inhibitor. Stimulation index=[insulin@20 mM glucose]/[insulin@2 mM glucose]. Relative Stim Index=Stim Index/Stim Index of Control.

FIG. 2A is a schematic illustrating the six stages of differentiation of human pluripotent stem cells to SC-β cells. hPSC=human pluripotent stem cell, DE=definitive endoderm cell, GT=gut tube cell, PP1=pancreatic progenitor cell 1, PP2=pancreatic progenitor cell 2, EN=endocrine progenitor cell, SC-β=stem cell-derived β cells.

FIG. 2B is a schematic illustrating an exemplary six step differentiation protocol for generating SC-β cells from pluripotent stem cells, as described further in Pagliuca et al. 2014 and PCT International Application No. PCT/US2014/041992.

FIG. 2C is a schematic illustrating an exemplary method for generating augmented SC-β cells according to the present invention, e.g., by contacting SC-β cells generated using the exemplary protocol shown in FIG. 2B with an effective amount of an AXL inhibitor and an antioxidant.

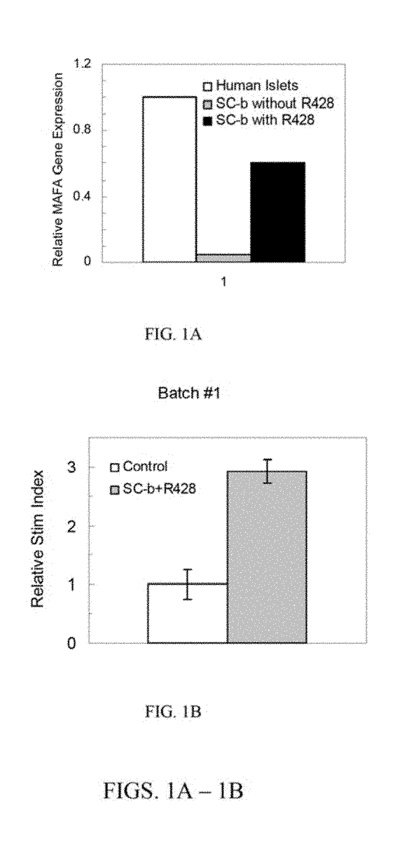

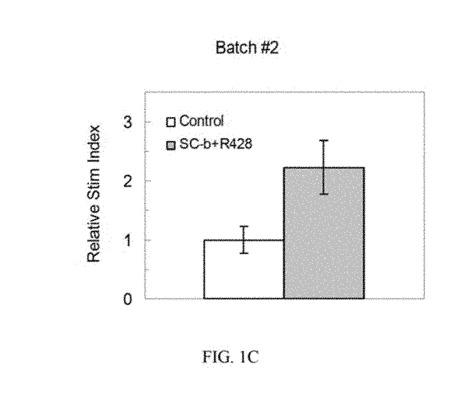

### DETAILED DESCRIPTION OF THE INVENTION

The present invention is directed to generating augmented SC-β cells, in particular augmented SC-β cells that exhibit improved in vitro and in vivo function. More particularly, work described herein demonstrates that augmented SC-β cells generated by contacting SC-β cells (or precursors thereof (e.g., endocrine progenitor cells) that are directed to differentiate into SC-β cells) with an agent that decreases the level and/or activity AXL receptor tyrosine kinase (AXL) (e.g., AXL inhibitor) and/or an antioxidant exhibit a greater stimulation index relative to SC-β cells generated using the same protocol but in the absence of contact with the AXL inhibitor and/or antioxidant. Additionally, augmented SC-β cells generated according to the methods of the present invention surprisingly and unexpectedly exhibit increased MAFA gene expression relative to SC-β cells (i.e., augmented SC-β cells exhibit approximately 12 times more MAFA gene expression than SC-β cells).

### Some Definitions

“Differentiation” is the process by which an unspecialized (“uncommitted”) or less specialized cell acquires the features of a specialized cell such as, for example, a pancreatic cell. A differentiated cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the lineage of a cell defines the heredity of the cell, i.e., which cells it came from and to what cells it can give rise. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

As used herein, “markers”, are nucleic acid or polypeptide molecules that are differentially expressed in a cell of interest. Differential expression means an increased level for a positive marker and a decreased level for a negative marker as compared to an undifferentiated cell. The detectable level of the marker nucleic acid or polypeptide is sufficiently higher or lower in the cells of interest compared to other cells, such that the cell of interest can be identified and distinguished from other cells using any of a variety of methods known in the art.

As used herein, a cell is “positive” or “+” for a specific marker (e.g., expresses the marker) when the specific marker is sufficiently detected in the cell. Similarly, the cell is “negative” or “−” for a specific marker when the specific marker is not sufficiently detected in the cell. For example, positive by FACS is usually greater than 2%, whereas the negative threshold by FACS is usually less than 1%.

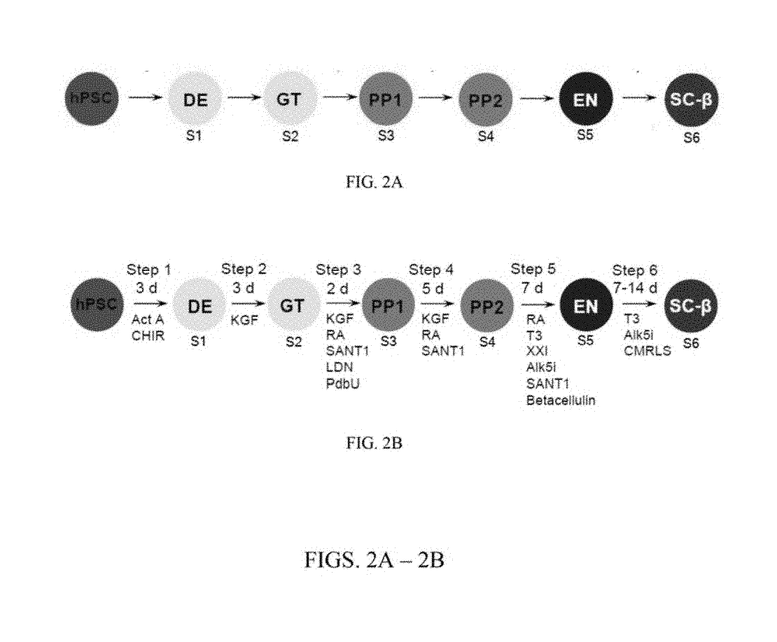

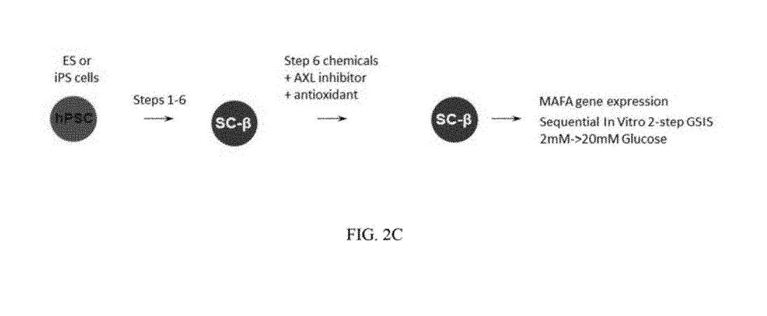

The process of differentiating pluripotent stem cells into functional pancreatic endocrine cells (i.e., SC-β cells) in vitro may be viewed in some aspects as progressing through six consecutive stages, as is shown in the exemplary protocol depicted in FIG. 2A. In this step-wise progression, “Stage 1” or “S1” refers to the first step in the differentiation process, the differentiation of pluripotent stem cells into cells expressing markers characteristic of definitive endoderm cells (“DE”, “Stage 1 cells” or “S1 cells”). “Stage 2” refers to the second step, the differentiation of cells expressing markers characteristic of definitive endoderm cells into cells expressing markers characteristic of gut tube cells (“GT”, “Stage 2 cells” or “S2 cells”). “Stage 3” refers to the third step, the differentiation of cells expressing markers characteristic of gut tube cells into cells expressing markers characteristic of pancreatic progenitor 1 cells (“PP1”, “Stage β cells” or “S3 cells”). “Stage 4” refers to the fourth step, the differentiation of cells expressing markers characteristic of pancreatic progenitor 1 cells into cells expressing markers characteristic of pancreatic progenitor 2 cells (“PP2”, “Stage 4 cells” or “S4 cells”). “Stage 5” refers to the fifth step, the differentiation of cells expressing markers characteristic of pancreatic progenitor 2 cells into cells expressing markers characteristic of pancreatic endoderm cells and/or pancreatic endocrine progenitor cells (“EN”, “Stage 5 cells” or “S5 cells”). “Stage 6” refers to the differentiation of cells expressing markers characteristic of pancreatic endocrine progenitor cells into cells expressing markers characteristic of pancreatic endocrine β cells (“SC-β cells”, “Stage 6 cells” or “S6 cells”). It should be appreciated, however, that not all cells in a particular population may progress through these stages at the same rate, i.e., some cells may have progressed less, or more, down the differentiation pathway than the majority of cells present in the population.

Characteristics of the various cell types associated with the stages shown in FIG. 2A are now described. “Definitive endoderm cells,” as used herein, refers to cells which bear the characteristics of cells arising from the epiblast during gastrulation and which form the gastrointestinal tract and its derivatives. Definitive endoderm cells express at least one of the following markers: FOXA2 (also known as hepatocyte nuclear factor 30 (“HNF3β”)), GATA4, SOX17, CXCR4, Brachyury, Cerberus, OTX2, goosecoid, C-Kit, CD99, and MIXL1. Markers characteristic of the definitive endoderm cells include CXCR4, FOXA2 and SOX17. Thus, definitive endoderm cells may be characterized by their expression of CXCR4, FOXA2 and SOX17. In addition, depending on the length of time cells are allowed to remain in Stage 1, an increase in HNF4α may be observed.

“Gut tube cells,” as used herein, refers to cells derived from definitive endoderm that can give rise to all endodermal organs, such as lungs, liver, pancreas, stomach, and intestine. Gut tube cells may be characterized by their substantially increased expression of HNF4α over that expressed by definitive endoderm cells. For example, a ten to forty fold increase in mRNA expression of HNF4α may be observed during Stage 2.

“Pancreatic progenitor 1 cells,” as used herein, refers to endoderm cells that give rise to the esophagus, lungs, stomach, liver, pancreas, gall bladder, and a portion of the duodenum. Pancreatic progenitor 1 cells express at least one of the following markers: PDX1, FOXA2, CDX2, SOX2, and HNF4α. Pancreatic progenitor 1 cells may be characterized by an increase in expression of PDX1, compared to gut tube cells. For example, greater than fifty percent of the cells in Stage 3 cultures typically express PDX1.

“Pancreatic progenitor 2 cells,” as used herein, refers to cells that express at least one of the following markers: PDX1, NKX6.1, HNF6, NGN3, SOX9, PAX4, PAX6, ISL1, gastrin, FOXA2, PTF1a, PROX1 and HNF4α. Pancreatic progenitor 2 cells may be characterized as positive for the expression of PDX1, NKX6.1, and SOX9.

“Pancreatic endocrine progenitor cells” or “endocrine progenitor cells” are used interchangeably herein to refer to pancreatic endoderm cells capable of becoming a pancreatic hormone expressing cell. Pancreatic endocrine progenitor cells express at least one of the following markers: NGN3; NKX2.2; NeuroD1; ISL1; PAX4; PAX6; or ARX. Pancreatic endocrine progenitor cells may be characterized by their expression of NKX2.2 and NeuroD1.

A “precursor thereof” as the term relates to a pancreatic endocrine progenitor cell refers to any cell that is capable of differentiating into a pancreatic endocrine progenitor cell, including for example, a pluripotent stem cell, a definitive endoderm cell, a gut tube cell, or a pancreatic progenitor cell, when cultured under conditions suitable for differentiating the precursor cell into the pancreatic pro endocrine cell.

“Pancreatic endocrine cells,” as used herein, refer to cells capable of expressing at least one of the following hormones: insulin, glucagon, somatostatin, ghrelin, and pancreatic polypeptide. In addition to these hormones, markers characteristic of pancreatic endocrine cells include one or more of NGN3, NeuroD1, ISL1, PDX1, NKX6.1, PAX4, ARX, NKX2.2, and PAX6. Pancreatic endocrine cells expressing markers characteristic of β cells can be characterized by their expression of insulin and at least one of the following transcription factors: PDX1, NKX2.2, NKX6.1, NeuroD1, ISL1, HNF30, MAFA and PAX6.

The terms “stem cell-derived β cell” and “SC-β cell” are used interchangeably herein to refer to non-native cells differentiated in vitro (e.g., from pluripotent stem cells) that display at least one marker indicative of a pancreatic β cell (e.g., PDX-1 or NKX6-1), express insulin, and display a GSIS response characteristic of an endogenous mature β cell both in vitro and in vivo. The GSIS response of the SC-β cells can be observed within two weeks of transplantation of the SC-β cell into a host (e.g., a human or animal). It is to be understood that SC-β cells need not be derived (e.g., directly) from stem cells, as the methods of the disclosure are capable of deriving SC-β cells from any endocrine progenitor cell that expresses insulin or precursor thereof using any cell as a starting point (e.g., one can use embryonic stem cells, induced-pluripotent stem cells, progenitor cells, partially reprogrammed somatic cells (e.g., a somatic cell which has been partially reprogrammed to an intermediate state between an induced pluripotent stem cell and the somatic cell from which it was derived), multipotent cells, totipotent cells, a transdifferentiated version of any of the foregoing cells, etc, as the invention is not intended to be limited in this manner). In some aspects, human cells are excluded that are derived from human embryonic stem cells obtained exclusively by a method necessitating the destruction of an embryo. The skilled artisan is well aware of such methods and how to avoid them for the purposes of generating augmented SC-β cells according to the methods of the present invention.

As used herein, an “augmented SC-β cell” refers to an SC-β cell that exhibits at least one characteristic that is enhanced or improved relative to a SC-β cell. As an example, the augmented SC-β cells of the present invention may exhibit increased MAFA gene expression relative to SC-β cells, and/or exhibit a greater stimulation index.

Used interchangeably herein are “d1”, “1d”, and “day 1”; “d2”, “2d”, and “day 2”, etc. These number letter combinations refer to a specific day of incubation in the different stages during the stepwise differentiation protocol of the instant application.

Methods for Generating SC-β Cells

As used herein, “directed to differentiate” refers to the process of causing a cell of a first cell type to differentiate into a cell of a second cell type. Recently, two protocols for directing the differentiation of pluripotent stem cells into insulin-producing endocrine cells that express key markers of mature pancreatic β cells (e.g., SC-β cells) have been reported, each of which includes differentiating cells into endocrine progenitor cells that can be directed to differentiate into SC-β cells, as well as protocols for directing the pancreatic endocrine progenitor cells into SC-β cells, which can be used in the method disclosed herein for generating augmented SC-β cells. First, as shown in FIG. 2B, an exemplary six-stage protocol for the large-scale production of functional human β cells using human pluripotent stem cells (hPSC) by sequential modulation of multiple signaling pathways in a three-dimensional cell culture system, without using any transgenes or genetic modification, was used to generate glucose-responsive, monohormonal insulin-producing cells that exhibited key β cell markers and β cell ultrastructure (see Pagliuca et al., 2014 and PCT International Application No. PCT/US2014/041992, both of which are incorporated herein by reference in their entirety). Pagliuca and colleagues reported that such cells mimicked the function of human islets in vitro and in vivo, and demonstrated the potential utility of such cells for in vivo transplantation to treat diabetes. Secondly, a seven-stage protocol that converts human embryonic stem cells (hESCs) into insulin-producing cells that expressed key markers of mature pancreatic β cells, such as MAFA, and displayed glucose-stimulated insulin secretion like that of human islets using static incubations in vitro was described (Rezania et al., 2014). Cells produced by such protocol, referred to as S7 cells, were found to rapidly reverse diabetics in mice within a little over a month.

Methods for Generating Augmented SC-β Cells

FIG. 2C is a schematic depicting an overview of an exemplary method for generating augmented SC-β cells in accordance with the present invention. In accordance with an example embodiment of the present invention, a method for generating augmented stem cell-derived β (SC-β) cells comprises contacting a cell population comprising SC-β cells, or precursors thereof, with an effective amount of an agent that decreases the level and/or activity of AXL receptor tyrosine kinase (AXL) and an antioxidant for a period of time sufficient for the level of MAFA gene expression to increase in the SC-β cells to a greater level than the level of MAFA gene expression in the SC-β cells in the absence of contact with the agent and the antioxidant, thereby generating augmented SC-β cells.

“Contacting”, “contacting the cell” and any derivations thereof as used herein, refers to any means of introducing an agent (e.g., nucleic acids, peptides, ribozymes, antibodies, small molecules, etc.) into a target cell or an environment in which the cell is present (e.g., cell culture), including chemical and physical means, whether directly or indirectly. Contacting also is intended to encompass methods of exposing a cell, delivering to a cell, or ‘loading’ a cell with an agent by viral or non-viral vectors, and wherein such agent is bioactive upon delivery. The method of delivery will be chosen for the particular agent and use. Parameters that affect delivery, as is known in the medical art, can include, inter alia, the cell type affected, and cellular location. In some aspects, contacting includes administering the agent to a subject. In some aspects, contacting refers to exposing a cell or an environment in which the cell is located (e.g., cell culture medium) to at least one agent that decreases the level and/or activity of AXL.

In some aspects, the cell precursors are selected from the group consisting of pluripotent stem cells, SOX17+ definitive endoderm cells, PDX1+ primitive gut tube cells, PDX1+/NKX6.1+ pancreatic progenitor cells, PDX1+/NKX6.1+/NEUROD1+ endocrine progenitor cells, PDX1+/NKX6.1+/NEUROD1+/insulin+/glucagon−/somatostatin− cells, and combinations thereof.

It is believed that augmented SC-β cells (or their cell precursors directed to differentiate into SC-β cells according to any suitable protocol) will exhibit improved in vitro and/or in vivo function when contacted with an agent that decreases the level and/or activity of AXL and/or an antioxidant. For example, work described herein demonstrates that augmented SC-β cells of the present invention exhibit an increased level of MAFA gene expression compared to SC-β cells. Those skilled in the art will appreciate that the extent of the increase in the level of MAFA gene expression in the augmented SC-β cells may depend on a variety of factors (e.g., the length of time the cells are exposed to the at least one agent (e.g., AXL inhibitor) and/or the antioxidant). The augmented SC-β cells of the present invention may exhibit a level of MAFA gene expression that is at least at least 2 fold, at least 2.1 fold, at least 2.2 fold, 2.3 fold, at least 2.4 fold, at least 2.5 fold, at least 2.6 fold, at least 2.7 fold, at least 2.8 fold, at least 2.9 fold, at least 3 fold, at least 4 fold, at least 5 fold, at least 6 fold, at least 7 fold, at least 8 fold, at least 9 fold, at least 10, at least 11 fold, at least 12 fold, at least 13 fold, at least 14 fold, at least 15 fold greater than the level of MAFA gene expression in a control SC-β cell. In some aspects, the level of MAFA gene expression in the augmented SC-β cells is at least 10 times greater than the level of MAFA gene expression in the SC-β cells.

The methods of the present invention contemplate contacting cells (e.g., SC-β cells or precursors thereof) with effective amounts of one or more agents that decrease the level and/or activity of AXL and/or an antioxidant. An “effective amount” of an agent (or composition containing such agent) refers to the amount sufficient to achieve a desired effect, e.g., when delivered to a cell or subject according to a selected administration form, route, and/or schedule. As will be appreciated by those of ordinary skill in this art, the absolute amount of a particular agent or composition that is effective may vary depending on such factors as the desired biological or pharmacological endpoint, the agent to be delivered, the target tissue, etc. Those of ordinary skill in the art will further understand that an “effective amount” may be contacted with cells or administered in a single dose, or the desired effect may be achieved by use of multiple doses. An effective amount of a composition may be an amount sufficient to reduce the severity of or prevent one or more symptoms or signs of a disorder (e.g., diabetes). In some aspects, the effective amount of the agent that decreases the level and/or activity of AXL comprises a concentration of between about 0.1 μM and about 110 μM. In some aspects, the effective amount of the agent comprises 1 μM. In some aspects, the effective amount of the agent comprises 2 μM. In some aspects, the effective amount of the agent comprises 3 μM. In some aspects, the SC-β cells (S6 cells) are contacted with 2 μM of R428 to generate augmented SC-β cells exhibiting an improved in vitro or in vivo function, and increased MAFA gene expression.

AXL Receptor Tyrosine Kinase (AXL)

AXL (Gene ID: 558; GenBank Accession: AAH32229.1; also known as ARK, UFO, JTK11 and Tyro7) is gene that encodes a receptor tyrosine kinase protein that is a member of the Tyro3-Axl-Mer (TAM) receptor tyrosine kinase subfamily. Receptor tyrosine kinases (RTKs) transmit extracellular signals by phosphorylating their protein partners on conserved tyrosine residues. AXL is a transmembrane receptor having a molecular weight between 100 and 140 kDA that includes an extracellular (N-terminal) domain and an intracellular (C-terminal) tyrosine kinase domain. The protein encoded by the gene contains an extracellular domain which is made of two N-terminal immunoglobulin-like motifs, followed by two fibronectin type-III motifs. AXL transduces extracellular matrix signals into the cytoplasm by binding to the vitamin K-dependent protein growth arrest-specific 6 (gas6). Generally, binding of Gas6 to the extracellular domain of AXL results in dimerization of Gas6/AXL complexes, and subsequently autophosphorylation of tyrosine residues on AXL's intracellular domain. Autophosphorylation of RTK is believed to increase phosphorylation activity b AXL substrates or formation of signaling complexes with phosphotryosine-binding domains. Three known autophosphorylation sites (Y779, Y821, and 7866) on AXL's intracellular domain exist, which are involved in binding AXL with subunits of phosphatidylinositol 3-kinase (PI3K), phospholipase C (PLC) and growth factor receptor-bound protein 2 (GRB2). AXL is known to interact with additional signaling molecules such as C1-TEN, NCK adaptor protein 2(Nck2), Ran binding protein in microtubule organizing centre (RanBPM), and suppressor of cytokine signaling 1 (SOCS-1). Activation of PI3K and its downstream target, serine/threonine protein kinase Akt (AKT), is an important step in AXL-dependent signal transduction. Signaling through the Gas6/AXL/PI3K/AKT pathway may protect cells from apoptosis. Akt activates ribosomal protein S6 kinase (S6K) of the mechanistic target of rapamycin (mTOR) pathway and phosphorylates BCL2-associated agonist of cell death (Bad), a pro-apoptotic protein. Akt inhibits pro-apoptotic caspase 3 and phosphorylates nuclear factor kappa-light-chain-enhancer of activated B cells (NF-κB) which increases expression of the anti-apoptotic proteins B-cell lymphoma 2 (Bcl-2) and B-cell lymphoma-extra large (Bcl-xL). Akt additionally phosphorylates αIIbβ3 integrins triggered by the Gas6/AXL pathway.

The present invention contemplates using any agent that decreases the level and/or activity of AXL (also referred to herein as a “AXL inhibitor”) in the methods for generating augmented SC-β cells. As used herein, “level” includes both mRNA expression levels and protein expression levels of AXL. As used herein, “activity” refers to any AXL performed by AXL in connection with its activation and/or signal transduction of any of its downstream targets (e.g., tyrosine kinase activity). As will be appreciated by the skilled artisan, kinase activity can be mentioned by assaying for phosphorylation of one or more AXL targets or substrates using methods available to the skilled artisan (e.g., tyrosine kinase assays).

In some aspects, the at least one agent (e.g., AXL inhibitor) decreases the level and/or activity of AXL by at least 1%, at least 2%, at least 3%, at least 4%, at least 5%, at least 10%, at least 15%, at least 20%, at least 25%, at least 30%, at least 40%, at least 50%, at least 60%, at least 70%, at least 80%, at least 90%, or at least 1.1 fold, at least 1.2 fold, 1.3 fold, at least 1.4 fold, at least 1.5 fold, at least 1.6 fold, at least 1.7 fold, at least 1.8 fold, at least 1.9 fold, at least 2 fold, at least 3 fold, at least 4 fold, at least 5 fold, at least 10 fold, at least 20 fold, at least 30 fold, at least 40 fold, at least 50 fold, or at least 100 fold, at least a 1,000 fold, at least 10,000 fold, or more in the cell or population of cells (e.g., SC-β cells) relative to the level and/or activity of AXL in cell or population of cells (e.g., SC-β cells) in the absence of contact with the at least one agent. In some aspects, the at least one agent completely abolishes the level and/or activity of AXL in the cell or population of cells (e.g., SC-β cells).

In some aspects, the at least one agent (e.g., AXL inhibitor) decreases the mRNA and/or protein expression level of AXL by at least 1%, at least 2%, at least 3%, at least 4%, at least 5%, at least 10%, at least 15%, at least 20%, at least 25%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, at least 99% or more in the cell or population of cells (e.g., augmented SC-β cells) relative to the mRNA and/or protein expression levels of AXL in the cell or population of cells (e.g., SC-β cells) in the absence of contact with the at least one agent. In some aspects, the at least one agent completely abolishes the mRNA and/or protein expression levels of AXL in the cell or population of cells (e.g., SC-β cells becoming augmented SC-β cells).

In some aspects, the at least one agent (e.g., AXL inhibitor) decreases the mRNA and/or protein expression level of AXL by at least 1%, at least 2%, at least 3%, at least 4%, at least 5%, at least 10%, at least 15%, at least 20%, at least 25%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, at least 99% or more in the cell or population of cells (e.g., SC-β cells) relative to the mRNA and/or protein expression levels of AXL in cell or population of cells (e.g., SC-β cells) in the absence of contact with the at least one agent. In some aspects, the at least one agent completely abolishes the mRNA and/or protein expression levels of AXL in the cell or population of cells (e.g., SC-β cells).

In some aspects, the at least one agent (e.g., AXL inhibitor) decreases phosphorylation of an AXL substrate or one of its downstream targets in the Gas6/AXL/PI3K/AKT pathway by at least 1%, at least 2%, at least 3%, at least 4%, at least 5%, at least 10%, at least 15%, at least 20%, at least 25%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, at least 99% or more in the cell or population of cells (e.g., SC-β cells) relative to the phosphorylation of an AXL substrate or one of its downstream targets in the cell or population of cells (e.g., SC-β cells) in the absence of contact with the at least one agent. In some aspects, the at least one agent completely abolishes the ability of AXL to phosphorylate one of its substrates and/or transduce signals.

AXL inhibitors can be small organic or inorganic molecules; saccharides; oligosaccharides; polysaccharides; biological macromolecules, e.g., peptides, proteins, and peptide analogs and derivatives; peptidomimetics; nucleic acids and nucleic acid analogs and derivatives (including but not limited to microRNAs, siRNAs, shRNAs, antisense RNAs, a ribozymes, and aptamers); an extract made from biological materials such as bacteria, plants, fungi, or animal cells; animal tissues; naturally occurring or synthetic compositions; and any combinations thereof.

Exemplary AXL inhibitors include, but are not limited to, an AXL fusion protein AXL tyrosine kinase inhibitor described in U.S. Pat. No. 8,168,415, an AXL kinase inhibitor described in U.S. Pat. No. 7,998,966, a diaminothiazole AXL inhibitor described in U.S. Pub. No. 2011/0092502, a pyrrolopyrimidinyl AXL kinase inhibitor described in U.S. Pub. No. 2010/0204221, an AXL antibody disclosed in U.S. Pub. No. 2010/0330095, a humanized AXL antibody disclosed in U.S. Pub. No. 2012/0117670, a quinolone derivative AXL kinase inhibitor disclosed in U.S. Pub. No. 2011/0092503, a triazole derivative AXL inhibitor disclosed in U.S. Pat. No. 8,389,557, an AXL modulator disclosed in U.S. Pub. No. 2009/0087431, an AXL receptor tyrosine kinase aptamer inhibitor having the sequence 5′AUGAUCAAUCGCCUCAAUUCGACAGGAGGCUCAC-3′ (SEQ ID NO: 1), as disclosed in U.S. Pat. No. 8,741,870, a substituted N-phenylpyrimidin-2-amine analog AXL inhibitor as described in U.S. Pat. No. 8,901,120, a polycyclic heteroaryl substituted triazole AXL inhibitor described in U.S. Pat. No. 8,741,898, and a bridged bicyclic heteroaryl substituted triazole AXL inhibitor described in U.S. Pat. No. 8,658,669.

In some aspects, the at least one agent comprises a microRNA (miR) that targets the 3′-UTR of the Axl gene e.g., miR-34a, miR-199a, and miR-199b as described in (Mudduluru et al. 2011).

In some aspects, the at least one agent comprises R428.

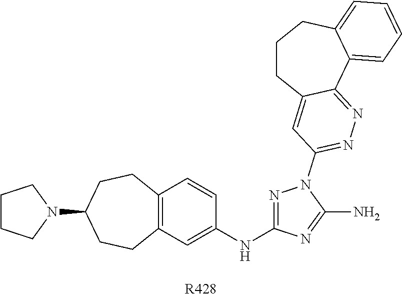

In some aspects, the at least one agent comprises a structural analog of R428.

In some aspects, the at least one agent comprises a 2,4,5-trisubstituted pyrimidine small organic molecule described in (Mollard et al. 2011), for example, a 5-alkylpyrimidine-based inhibitor derivable from Table 1 of Mollard et al. below, and/or an Amide- and/or Sulfonamide-containing inhibitor derivable from Table 2 of Mollard et al. below.

TABLE 1
Structure—Activity Relationship and Docking Parameters for 5-Alkylpyrimidine-Based Inhibitors

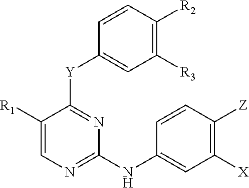

| | | | | | | | Binding energy | AXL kinase |
|---|---|---|---|---|---|---|---|---|
| Compound | R₁ | R₂ | R₃ | X | Y | Z | (kcal/mol) | IC₅₀ (μM) |
| 3 | Me | F | Cl | F | bond | Z₁ | −26.67 | 2.80 |
| 4 | Me | F | H | F | bond | Z₁ | −31.24 | 6.10 |
| 5 | Me | F | Cl | H | bond | Z₂ | −38.24 | 0.73 |
| 6 | Me | F | Cl | H | bond | Z₃ | −21.81 | 1.63 |
| 7 | Me | F | Cl | H | bond | Z₄ | −34.26 | 0.42 |
| 8 | Me | H | Cl | H | bond | Z₂ | −36.89 | 0.44 |
| 9 | Me | CMe₂CN | H | H | bond | Z₂ | −34.89 | 0.29 |
| 10 | Me | F | Cl | H | O | Z₂ | −27.49 | 1.40 |
| 11 | Me | F | Cl | H | NH | Z₂ | −19,.21 | 62.7 |
| 12 | Cyclopropyl | F | Cl | H | bond | Z₂ | −26.92 | 3.40 |

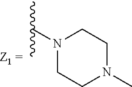 [SMILES: *N1CCN(C)CC1]

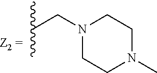 [SMILES: *CN1CCN(C)CC1]

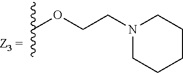 [SMILES: *OCCN1CCCCC1]

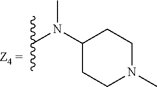 [SMILES: *N(C)C1CCN(C)CC1]

TABLE 2
Structure—Activity Relationship and Binding Energy Terms for Amide- and Sulfonamide-Containing Inhibitors

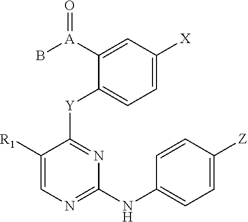

| | | | | | | | | | Cell viability |
|---|---|---|---|---|---|---|---|---|---|
| | | | | | | | Binding energy | AXL kinase | IC50 (μM) |
| Compd | R₁ | A | B | X | Y | Z | (kcal/mol) | IC₅₀ (μM) | PSN-1 |
| 13 | Cl | S═O | B₁ | H | NH | Z₂ | −36.63 | 0.027 | 0.006 |
| 14 | Cl | C | B₁ | H | NH | Z₂ | −33.29 | 0.088 | 0.070 |
| 15 | Me | S═O | B₂ | F | bond | Z₂ | −29.14 | 2.93 | N.D. |
| 16 | F | S═O | B₂ | F | bond | Z₂ | −29.67 | 1.15 | N.D. |
| 17 | CF₃ | C | B₁ | H | NH | Z₂ | −26.21 | >10 | N.D. |
| 18 | Cl | C | B₂ | H | NH | Z₂ | −38.12 | 0.061 | 0.021 |
| 19 | Cl | C | B₃ | H | NH | Z₂ | −39.21 | 0.032 | 0.064 |
| 20 | Cl | S═O | B₂ | H | NH | Z₂ | −49.89 | 0.019 | 0.002 |
| 21 | Cl | S═O | B₄ | F | NH | Z₂ | −41.26 | 0.240 | N.D. |
| 22 | Cl | S═O | B₂ | F | NH | Z₂ | −37.87 | 0.037 | 0.009 |
| 23 | Cl | S═O | B₂ | Cl | NH | Z₂ | −28.21 | 1.08 | N.D. |
| 24 | Cl | S═O | B₂ | OH | NH | Z₂ | −29.21 | 1.32 | N.D. |
| 25 | Cl | S═O | B₂ | F | NMe | Z₂ | −26.93 | 3.91 | N.D. |
| 26 | Cl | S═O | B₁ | F | bond | Z₂ | −41.87 | 0.082 | N.D. |
| 27 | Cl | S═O | B₂ | F | bond | Z₂ | −40.96 | 0.091 | 0.192 |
| 28 | Cl | S═O | B₁ | H | NH | Z₃ | −36.84 | 0.056 | 0.410 |
| 29 | Cl | S═O | B₁ | H | NH | Z₁ | −29.34 | 1.73 | N.D. |

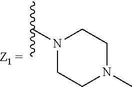 [SMILES: *N1CCN(C)CC1]

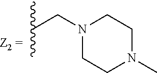 [SMILES: *CN1CCN(C)CC1]

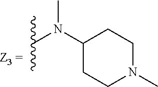 [SMILES: *N(C)C1CCN(C)CC1]

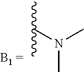 [SMILES: *N(C)C]

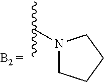 [SMILES: *N1CCCC1]

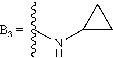 [SMILES: *NC1CC1]

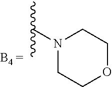 [SMILES: *N1CCOCC1]

In some aspects, the at least one agent comprises AZ.

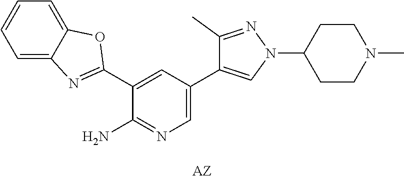 [SMILES: Cc1nn(C2CCN(C)CC2)cc1-c1cnc(N)c(-c2nc3ccccc3o2)c1]

In some aspects, the at least one agent comprises Max-Planck-Gesellschaft AXL inhibitor.

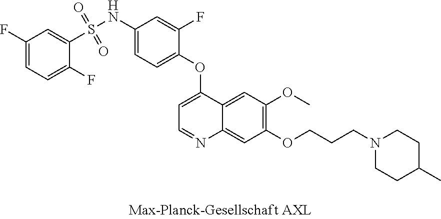 [SMILES: COc1cc2c(Oc3ccc(NS(=O)(=O)c4cc(F)ccc4F)cc3F)ccnc2cc1OCCCN1CCC(C)CC1]

inhibitor

In some aspects, the at least one agent comprises SuperGen.

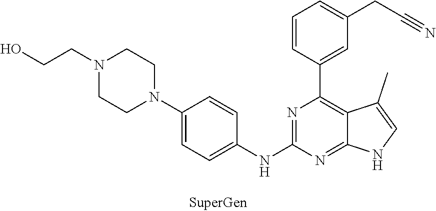 [SMILES: Cc1c[nH]c2nc(Nc3ccc(N4CCN(CCO)CC4)cc3)nc(-c3cccc(CC#N)c3)c12]

In some aspects, the at least one agent comprises BMS-777607.

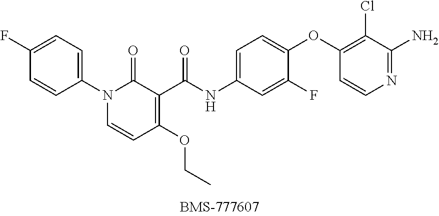 [SMILES: CCOc1ccn(-c2ccc(F)cc2)c(=O)c1C(=O)Nc1ccc(Oc2ccnc(N)c2Cl)c(F)c1]

In some aspects, the at least one agent comprises PF-02341066.

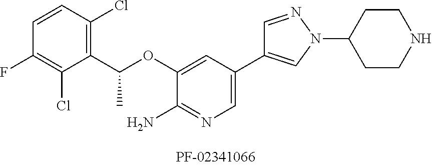 [SMILES: C[C@@H](Oc1cc(-c2cnn(C3CCNCC3)c2)cnc1N)c1c(Cl)ccc(F)c1Cl]

In some aspects, the at least one agent comprises a Janssen AXL inhibitor.

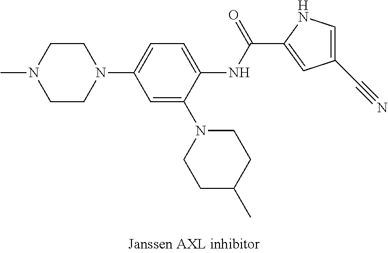 [SMILES: CC1CCN(c2cc(N3CCN(C)CC3)ccc2NC(=O)c2cc(C#N)c[nH]2)CC1]

In some aspects, the at least one agent comprises Compound-52.

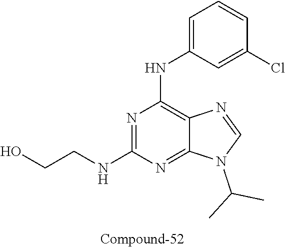 [SMILES: CC(C)n1cnc2c(Nc3cccc(Cl)c3)nc(NCCO)nc21]

In some aspects, the at least one agent comprises an agent that decreases the level and/or activity of Gas6 (e.g., by interfering with Gas6 binding to an extracellular domain of AXL).

Antioxidants

The present invention contemplates using any antioxidant that when used in combination with the at least one agent (e.g., AXL inhibitor) generates augmented SC-β cells that exhibit increased MAFA gene expression levels relative to the MAFA gene expression levels of control SC-β cells which were not contacted at the appropriate time with the at least one agent and the antioxidant.

Exemplary antioxidants include, but are not limited to, N-acetylcysteine, ascorbic acid, vitamin E, disodium 4,5-dihydroxy-1,3-benzenedisulfonate (Tiron).

The effective amount of antioxidant contemplated for use in the methods of generating augmented SC-β cells comprises a concentration range of between 0.1 mM and 10 mM. In accordance with aspects of the present invention, the effective amount of the antioxidant comprises a concentration of 1 mM. In accordance with aspects of the present invention, the effective amount of the antioxidant comprises a concentration of 2 mM. In accordance with aspects of the present invention, the effective amount of the antioxidant comprises a concentration of 3 mM.

In accordance with aspects of the present invention, the antioxidant comprises N-acetylcysteine. The effective amount of N-acetylcysteine contemplated for use in the methods of generating augmented SC-β cells comprises a concentration range of between 0.1 mM and 10 mM. In accordance with aspects of the present invention, the effective amount of the N-acetylcysteine comprises a concentration of 1 mM. In accordance with aspects of the present invention, the effective amount of the N-acetylcysteine comprises a concentration of 2 mM. In accordance with aspects of the present invention, the effective amount of the N-acetylcysteine comprises a concentration of 3 mM.

The methods for generating augmented SC-β cells contemplate contacting SC-β cells for a period of time sufficient for the level of MAFA gene expression to increase to a greater level (e.g., at least 1.1 fold greater, at least 1.2 fold greater, at least 1.3 fold greater, at least 1.4 fold greater, at least 1.5 fold greater, at least 1.6 fold greater, at least 1.7 fold greater, at least 1.8 fold greater, at least 1.9 fold greater, at least 2.0 fold greater, at least 2.1 fold greater, at least 2.2 fold greater, at least 2.3 fold greater, at least 2.4 fold greater, at least 2.5 fold greater, at least 2.6 fold greater, at least 2.7 fold greater, at least 2.8 fold greater, at least 2.9 fold greater, at least 3.0 fold greater, at least 4.0 fold greater, at least 5.0 fold greater, at least 6.0 fold greater, at least 7 fold greater) than the level of MAFA gene expression in the SC-β cells in the absence of contact with the agent and/or the antioxidant.

Those skilled in the art will appreciate that the period of time may vary depending on a variety of factors (e.g., augmented SC-β cells may exhibit greater levels of MAFA expression and greater stimulation indices when exposed to the at least one agent and antioxidant for greater periods of time). In accordance with aspects of the present invention, the period of time comprises between 7 days and 21 days. For example, MAFA gene expression was observed to increase to a level that is 12 times greater than the level of MAFA gene expression in as little as 2 weeks (14 days). In accordance with aspects of the present invention, the period of time comprises 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days or longer.

It should be appreciated that the population of cells comprising the SC-β cells (or precursor cells thereof) contacted in accordance with the method may comprise different cells types as the cells are progressing toward augmented SC-β cells. Preferably, a maximum amount of SC-β cells in the population contacted with the at least one agent (e.g., AXL inhibitor) and/or antioxidant become augmented SC-β cells. In some aspects, between at least 5% and 65% of the SC-β cells in the population become augmented SC-β cells.

SC-β Cells Obtained by the Method of Generating SC-β Cells

In accordance with an embodiment of the present invention, an isolated augmented SC-β cell or population thereof generated according to a method described herein is provided. The isolated augmented SC-β cell or population exhibits a GSIS response both in vitro and in vivo. The isolated augmented SC-β cell or population also exhibits at least one characteristic feature of a mature endogenous β cell (e.g., monohormonality). In some aspects, an isolated augmented SC-β cell or population thereof exhibits a stimulation index that is at least between 2.3-fold and 2.9-fold greater than the stimulation index of a control SC-β cell. In some aspects, an isolated augmented SC-β cell or population thereof produces between approximately 300 uIU to about 4000 uIU per 30 minute per 10⁶ total cells incubation at a high glucose concentration.

The augmented SC-β cells disclosed herein share many distinguishing features of native β cells, but are different in certain aspects (e.g., gene expression profiles). In some embodiments, the augmented SC-β cell is non-native. As used herein, “non-native” means that the augmented SC-β cells are markedly different in certain aspects from β cells which exist in nature, i.e., native β cells. It should be appreciated, however, that these marked differences typically pertain to structural features which may result in the augmented SC-β cells exhibiting certain functional differences, e.g., although the gene expression patterns of augmented SC-β cells differs from native β cells, the augmented SC-β cells behave in a similar manner to native β cells but certain functions may be altered (e.g., improved) compared to native β cells. For example, a higher frequency of augmented SC-β cells respond to 20 mM glucose compared to the frequency of native β cells. Other differences between augmented SC-β cells and native β cells would be apparent to the skilled artisan based on the data disclosed herein.

The augmented SC-β cells of the disclosure share many characteristic features of β cells which are important for normal β cell function. For example, the augmented SC-β cells (e.g., human) generated according to the methods described herein may exhibit at least one of the following characteristics of an endogenous mature pancreatic β cell: i) a response to multiple glucose challenges that resembles the response of endogenous islets (e.g., at least one, at least two, or at least three or more sequential glucose challenges); ii) a morphology that resembles the morphology of an endogenous β cell; iii) packaging of insulin into secretory granules or encapsulated crystalline insulin granules; iv) a stimulation index of greater than at least 1.4; v) cytokine-induced apoptosis in response to cytokines; vi) enhanced insulin secretion in response to known antidiabetic drugs (e.g., secretagogues); vii) monohormonal, i.e., they do not abnormally co-express other hormones, such as glucagon, somatostatin or pancreatic polypeptide; viii) a low rate of replication; and ix) increased intracellular Ca²⁺ in response to glucose.

In accordance with an embodiment of the present invention, a microcapsule comprising the isolated augmented SC-β cell or population thereof encapsulated therein is provided.

In accordance with an embodiment of the present invention, a macroencapsulation device comprising the isolated augmented SC-β cell or population thereof is provided.

In accordance with an embodiment of the present invention, a cell line comprising an isolated augmented SC-β cell that stably expresses insulin is provided.

Assays

In accordance with an embodiment of the present invention, an isolated augmented SC-β cell or population thereof generated according to the methods herein, or an augmented SC-β cell that stably expresses insulin, can be used in various assays. In some aspects, an isolated augmented SC-β cell, population thereof, or an augmented SC-β cell that stably expresses insulin, can be used in an assay to identify one or more candidate agents which promote or inhibit a β cell fate selected from the group consisting of β cell proliferation, β cell replication, β cell death, 0 cell function, β cell susceptibility to immune attack, and β cell susceptibility to dedifferentiation or differentiation. In some aspects, an isolated augmented SC-β cell, population thereof, or an augmented SC-β cell that stably expresses insulin, can be used in an assay to identify one or more candidate agents which promote the differentiation of at least one insulin-positive endocrine cell or a precursor thereof into at least one SC-β cell. The assays typically involve contacting the isolated augmented SC-β cell, population thereof, or an augmented SC-β cell that stably expresses insulin, with one or more candidate agents to be assessed for its ability to i) promote or inhibit a β cell fate selected from the group consisting of β cell proliferation, β cell replication, β cell death, β cell function, β cell susceptibility to immune attack, and β cell susceptibility to dedifferentiation or differentiation, or ii) promoting the differentiation of at least one insulin-positive endocrine cell or a precursor thereof into at least one SC-β cell and assessing whether the candidate agent possesses the ability to i) promote or inhibit a β cell fate selected from the group consisting of β cell proliferation, β cell replication, β cell death, β cell function, β cell susceptibility to immune attack, and β cell susceptibility to dedifferentiation or differentiation, or ii) promoting the differentiation of at least one insulin-positive endocrine cell or a precursor thereof into at least one SC-β cell.

Methods for Treatment

In accordance with an embodiment of the present invention, methods for the treatment of a subject in need thereof are provided. The methods entail administering to a subject in need thereof an isolated population of augmented SC-β cells, a microcapsule comprising augmented SC-β cells encapsulated therein, and/or a macroencapsulation device comprising the augmented SC-β cells encapsulated therein. In some aspects, the subject is in need of additional β cells. In some aspects, the subject has, or has an increased risk of developing diabetes. An augmented SC-β cell or population (e.g., isolated) of augmented SC-β cells generated by a method of the present invention can be administered to a subject for treatment of type 1 or type 2 diabetes. In some aspects, the subject has, or has an increased risk of developing, a metabolic disorder. In some aspects, administering to the subject comprises implanting augmented SC-β cells, a microcapsule comprising augmented SC-β cells, or a macroencapsulation device comprising augmented SC-β cells into the subject. The subject may be a human subject or an animal subject. In some aspects, the cells may be implanted as dispersed cells or formed into clusters that may be infused into the hepatic portal vein. In some aspects, cells may be provided in biocompatible degradable polymeric supports, porous non-degradable devices or encapsulated to protect from host immune response. Cells may be implanted into an appropriate site in a recipient. The implantation sites include, for example, the liver, natural pancreas, renal subcapsular space, omentum, peritoneum, subserosal space, intestine, stomach, or a subcutaneous pocket.

To enhance further differentiation, survival or activity of the implanted cells in vivo, additional factors, such as growth factors, antioxidants or anti-inflammatory agents, can be administered before, simultaneously with, or after the administration of the cells. These factors can be secreted by endogenous cells and exposed to the administered cells in situ. Implanted cells can be induced to differentiate by any combination of endogenous and exogenously administered growth factors known in the art.

The amount of cells used in implantation depends on a number of various factors including the patient's condition and response to the therapy, and can be determined by one skilled in the art.

In some aspects, the method of treatment further comprises incorporating the cells into a three-dimensional support prior to implantation. The cells can be maintained in vitro on this support prior to implantation into the patient. Alternatively, the support containing the cells can be directly implanted in the patient without additional in vitro culturing. The support can optionally be incorporated with at least one pharmaceutical agent that facilitates the survival and function of the transplanted cells.

Artificial Islet or Pancreas

In accordance with an embodiment of the present invention, an artificial islet or pancreas is provided. The artificial islet or pancreas can be constructed using the augmented SC-β cells generated according to the methods described herein.

An artificial pancreas is a device that encapsulates and nurtures islets of Langerhans to replace the islets and β cells destroyed by type 1 diabetes. An artificial pancreas may contain a million islets or more, and may be implanted in the peritoneal cavity or under the skin where it can respond to changing blood glucose levels by releasing hormones, such as insulin. An artificial pancreas may be made using living (e.g., glucose-sensing and insulin secreting islets) and nonliving components (e.g., to shield the islets from the diabetic's body and its destructive immune mechanism while permitting the islets to thrive).

The present invention contemplates using β cells in any artificial pancreas. In some aspects, the artificial pancreas comprises microencapsulated or coated islets comprising augmented SC-β cells generated according to the methods herein. In some aspects, the artificial pancreas comprises a macroencapsulation device into which islet cells comprising augmented SC-β cells generated according to the methods herein are grouped together and encapsulated. In some aspects, the macroencapsulation device comprises a PVA hydrogel sheet for an artificial pancreas of the present invention (Qi et al., 2004). In some aspects, the artificial islet comprises augmented SC-β cells generated according to the methods herein, along with other islet cells (α, δ, etc.) in the form of an islet sheet. The islet sheet comprises a layer of artificial human islets comprising the augmented SC-β cells macroencapsulated within a membrane (e.g., of ultra-pure alginate). The sheet membrane is reinforced with mesh and may be coated on the surface to prevent or minimize contact between the cells encapsulated inside and the transplantation recipient's host immune response. Oxygen, glucose, and other nutrients readily diffuse into the sheet through the membrane nurturing the islets, and hormones, such as insulin readily diffuse out. Additional examples of membranes designed for macroencapsulation/implantation of an artificial islet or pancreas can be found in the literature (Isayeva et al. 2003). Another example of a macroencapsulated implant suitable for an artificial islet or pancreas can be found in the literature (Aurelien, et al. 2014).

### Terminology

The articles “a”, “an” and “the” as used herein, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in haec verba herein. It should also be understood that any embodiment of the invention, e.g., any embodiment found within the prior art, can be explicitly excluded from the claims, regardless of whether the specific exclusion is recited in the specification. For example, any agent may be excluded from the genus of AXL inhibitors or antioxidants claimed.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments 5% of a number in either direction (greater than or less than the number) unless otherwise stated or otherwise evident from the context (except where such number would impermissibly exceed 100% of a possible value).

Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group.

Certain claims are presented in dependent form for the sake of convenience, but any dependent claim may be rewritten in independent format to include the limitations of the independent claim and any other claim(s) on which such claim depends, and such rewritten claim is to be considered equivalent in all respects to the dependent claim (either amended or unamended) prior to being rewritten in independent format. It should also be understood that, unless clearly indicated to the contrary, in any methods claimed herein that include more than one act, the order of the acts of the method is not necessarily limited to the order in which the acts of the method are recited, but the invention includes embodiments in which the order is so limited. It is contemplated that all aspects described above are applicable to all different embodiments of the invention. It is also contemplated that any of the above embodiments can be freely combined with one or more other such embodiments whenever appropriate.

### REFERENCES

1. Bellin et al., (2012). Potent induction immunotherapy promotes long-term insulin independence after islet transplantation in type 1 diabetes. Am. J. Transplant. 12, 1576-1583.
2. Pagliuca et al. (2014). Generation of Functional Human Pancreatic β cells In Vitro. Cell. 159, 428-439.
3. Rezania et al. (2014). Reversal of diabetes with insulin-producing cells derived in vitro from human pluripotent stem cells. Nat. Biotech. 32(11), 1121-1133.
4. Isayeva, et al. (2003). Characterization and performance of membranes designed for macroencapsulation/implantation of pancreatic islet cells.

Biomaterials 24(20), 3483-3491.
5. Motte, et al. (2014). Composition and function of macroencapsulated human embryonic stem cell-derived implants: comparison with clinical human islet cell grafts. American Journal of Physiology-Endocrinology and Metabolism 307(9), E838-E846.
6. Qi et al. (2004). PVA hydrogel sheet macroencapsulation of the bioartificial pancreas. Biomaterials 24(27), 5885-5892.
7. Mudduluru et al. (2011). Regulation of Axl receptor tyrosine kinase expression by miR-34a and miR-199a/b in solid cancer. Oncogene 30(25) 2889-2899.
8. Mollard et al. (2011). Design, Synthesis, and Biological Evaluation of a Series of Novel AXL Kinase Inhibitors. ACSMedicinal Chemistry Letters 2, 907-912.

What is claimed is:

1. A method for generating augmented stem cell-derived β (SC-β) cells, the method comprising contacting a cell population comprising SC-β cells, or precursors thereof, with an effective amount of an agent that decreases the level and/or activity of AXL receptor tyrosine kinase (AXL) and an antioxidant for a period of time sufficient for the level of MAFA gene expression to increase in the SC-β cells to at least 2 fold greater than the level of MAFA gene expression in the SC-β cells in the absence of contact with the agent and the antioxidant, thereby generating augmented SC-β cells.

2. The method of claim 1, wherein the level of MAFA gene expression in the augmented SC-β cells is at least 10 times greater than the level of MAFA gene expression in the SC-β cells.

3. The method of claim 1, wherein the cell precursors are selected from the group consisting of pluripotent stem cells, SOX17+ definitive endoderm cells, PDX1+ primitive gut tube cells, PDX1+/NKX6.1+ pancreatic progenitor cells, PDX1+/NKX6.1+/NEUROD1+ endocrine progenitor cells, PDX1+/NKX6.1+/NEUROD+/insulin+/glucagon−/somatostatin− cells, and combinations thereof.

4. The method of claim 1, wherein the agent comprises R428.

5. The method of claim 1, wherein the effective amount of the agent comprises a concentration of 2 μM.

6. The method of claim 1, wherein the antioxidant is selected from the group consisting of N-acetylcysteine, ascorbic acid, vitamin E, disodium 4,5-dihydroxy-1,3-benzenedisulfonate (Tiron).

7. The method of claim 1, wherein the effective amount of the antioxidant comprises a concentration of 1 mM.

8. The method of claim 1, wherein the period of time comprises between 7 days and 21 days.

9. The method of claim 1, wherein between at least 5% and 65% of the SC-β cells in the population become augmented SC-β cells.

10. An isolated augmented non-native SC-β cell or population thereof that exhibits a glucose stimulated insulin secretion (GSIS) response both in vitro and in vivo.

11. An isolated augmented SC-β cell or population thereof according to claim 10 that exhibits a stimulation index that is at least between 2.3-fold and 2.9-fold greater than the stimulation index of a control SC-β cell.

12. An isolated augmented SC-β cell or population thereof according to claim 10 that produces between approximately 300 uIU and 4000 uIU per 30 minute incubation at a high glucose concentration.

13. A microcapsule comprising the isolated augmented SC-β cell or population thereof according to claim 10 encapsulated therein.

14. A microencapsulation device comprising the isolated augmented SC-β cell or population thereof according to claim 10 encapsulated therein.

15. A cell line comprising the isolated augmented SC-β cell of claim 10, wherein the cell line stably expresses insulin.

16. An assay comprising the isolated augmented SC-β cell or population thereof according to claim 10, for use in: i) identifying one or more candidate agents which promote or inhibit a β cell fate selected from the group consisting of β cell proliferation, β cell replication, β cell death, β cell function, β cell susceptibility to immune attack, and β cell susceptibility to dedifferentiation or differentiation; or ii) identifying one or more candidate agents which promote the differentiation of at least one insulin-positive endocrine cell or a precursor thereof into at least one SC-β cell.

17. A method for the treatment of a subject in need thereof, the method comprising administering to a subject in need thereof an isolated population of augmented SC-β cells produced according to the methods of claim 1.

18. An artificial islet or pancreas comprising augmented SC-β cells produced according to the method claim 1.
